(19) United States
(12) Patent Application Publication
(10) Pub. No.: US 2016/0185792 A1
(43) Pub. Date: Jun. 30, 2016

(54) METHOD FOR THE PURIFICATION OF EPOTHILONES VIA CRYSTALLIZATION

(71) Applicant: SANDOZ AG, Basel (CH)

(72) Inventor: Thorsten WILHELM, Kundl/Tirol (AT)

(21) Appl. No.: 14/911,402

(22) PCT Filed: Aug. 18, 2014
(86) PCT No.: PCT/EP2014/067528

§ 371 Date: Feb. 10, 2016

(30) Foreign Application Priority Data
Aug. 19, 2013 (EP) 13180858.6

(51) Int. Cl. C07D 493/04

(52) U.S. Cl. CPC C07D 493/04

(57) ABSTRACT

The present invention provides a new method of separating epothilones from one another that can be used on an industrial scale for the selective enrichment of epothilone and to provide a crystalline solid of enhanced purity, which can be used for the production of pharmaceutical preparations without using normal or reverse-phase chromatography or any energy input via distillation apparatus, wherein the new method comprises the steps:
a. dissolving a crude containing epothilones in an nonpolar protic solvent,
b. adding an amount of a polar protic anti-solvent to form a slurry,
c. isolating a crystalline solid from the slurry.

The present invention relates to a new method of separating epothilones from one another that can be used on an industrial scale and its usage in the production of pharmaceutical preparations.

Epothilones represent a relatively new class of microtubule-targeting agents that mimic the biological effects of taxanes. In contrast to taxanes, these macrolactone compounds are also active in vitro against multidrug-resistant cancer cell lines. The six naturally occurring epothilones A to F have been identified and characterized to date (Altmann 2003, Mini Rev Med Chem. 2003, 3(2), 149-58.). For example, epothilones A and B, which nowadays find utility in the pharmaceutical field, having the structures:

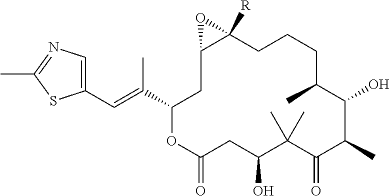

wherein R signifies hydrogen (epothilone A) or methyl (epothilone B).

Due to its biological activity, epothilines have been the target of many chemical total syntheses, which at the same time can provide a high degree of purity.

Danishefsky et al. (Angew. Chem. 1996, 108(23-24), 2976) discloses the first total synthesis of epothilone B, achieved via an extension Suzuki coupling reaction and a subsequent stereoselective epoxidation with dimethyldioxirane. Taylor and Chen (Org. Lett. 2001, 3(14), 2221-2224) disclose a total synthesis of epothilone B and D, wherein the route is highlighted by a final epoxidation of epothilone D yielding epothilone B in 65% yield, containing unconverted epothilone D.

Despite the success of epothilone total synthesis, these efforts are tedious, time-consuming, and expensive.

Due to their anti-fungal properties, epothilones were initially investigated as plant protective agents, which were originally obtained by fermentation as secondary metabolite of myxobacteria. Herein, the strain Sorangium (S.) cellulosum was found to produce and secreted the epothilone forms A and B.

WO 9310121 A1 discloses a method for cultivation of S. cellulosum in a fermentation medium containing carbon sources, nitrogen sources and mineral salts. During the fermentation process various synthesized epothilones are bound to an adsorber resin. The bound epothilones were separated by reverse-phase chromatography and crystallized in toluene/ethyl acetate or ethyl acetate.

When isolated in the pure form, epothilones A and B showed broad cytotoxic activity against eukaryotic cells and selectivity against breast and colon tumor cell lines.

US 2007/0122891 A1 discloses methods to improve the ratio of epothilone B to A produced by S. cellulosum, by adding a propionate as additive to the fermentation media. US 2007/0122891 A1 further discloses a process for the purification of epothilone B from the fermentation media, achieved by a combination of chromatography and crystallization or by crystallization only. The crystallization is carried out by extracting a resin, containing epothilones with a first distillative solvent, preferably ethyl acetate and swap it with a second solvent, characterized by a high boiling point, wherein the distillative solvent is distilled away to allow crystallization of epothilone B.

Disadvantageously, the high energy input during distillative processes easily results in decomposition of epothilone B, which carries a thermolabile epoxide ring structures.

EP 1 428 826 A2 discloses a method of separating epothilones, especially epothilone A and B from another, which is characterized by chromatography on a reversed-phase column using an acetonitrile/water mixture as an eluent.

Reverse-phase chromatography has enjoyed widespread acceptance as a rapid, moderate purification technique. However it suffers from the great disadvantages in consumption high amounts of solvents and high costs for equipment. Furthermore, reverse-phase chromatography is very difficult to handle and unsuitable for large-scale production.

The invention has the object of finding a method for the purification of epothilones, especially epothilone B for large-scale industrial application, which has significant potential to save primary energy and reduce emissions. In particular the method shall reduce the amounts of solvents needed for purification.

The objective of the invention is solved by a method of separating epothilones from another, preferably from a mixture containing epothilone B, with the following steps:
a. dissolving a crude containing epothilones in an nonpolar protic solvent, preferably an alkyl cyanide,
b. adding an amount of a polar protic anti-solvent, preferably water, in a ratio to the amount of said nonpolar protic solvent of 12:1 to about 1:3 (volume/volume) to form a slurry,
c. isolating a crystalline solid, preferably containing epothilone B, from the said slurry.

Advantageously the invention enables a purification of epothilones from impurities via crystallization without using normal or reverse-phase chromatography or any energy input via distillation apparatus.

The general terms used hereinabove and herein below preferably have the meanings given herein below:

Where reference is made hereinabove and herein below to documents, these are incorporated insofar as is necessary.

The crude containing epothilones is preferably obtained as fermentation product and/or by chemical synthesis. A preferred fermentation is performed in S. cellulosum, wherein the cultivation conditions of the recombinantly produced microorganisms are known by those skilled in the art.

A crude containing epothilones can also be obtained by a chemical total synthesis, such as those disclosed by Meng et al. (I. Am. Chem. Soc. 1996, 119(42), 10073-10092); Nicolaou et al. (J. Am. Chem. Soc. 1997, 119(34), 7974-7991) and Schinzer et al. (Chem. Eur. J. 1999, 5(9), 2483-2491).

The epothilones occurring in a crude are preferably epothilone A and/or B, but also other epothilones, for example epothilones C and D named in International Application WO 97/19086 and WO 98/22461, epothilones E and F named in WO 98/22461, and further epothilones obtainable from corresponding microorganisms.

In some embodiments, the epothilones are selected from ixabepilone, epothilone B, epothilone D, BMS310705, dehydelone and ZK-EPO.

As used herein, the term “crude containing epothilones” refers to a mixture containing epothilone B and at least another epothilone selected from epothilones A, C, D, E, F, ixabepilone, BMS310705, dehydelone and ZK-EPO, which is in a substantially unrefined state. The crude containing epothilones is preferably obtained as fermentation product or alternatively by chemical synthesis or a combination thereof.

Epothilones according to the invention are macrolactones characterized by a relatively low content of functional groups.

As used herein, molecules of aprotic (nonpolar) solvents lack an acidic hydrogen atom and having a moderate dielectric constant, preferably between 0 and 55, more preferably between 5 and 50. In particular the term “aprotic solvents” in particular means those solvents identified as aprotic solvents in Reichardt, Solvents and Solvent Effects in Organic Chemistry (VCH Verlagsgesellschaft mbH, Weinheim, Germany, 1990).

Preferably aprotic solvents have moderate electric dipole moments above 2, more preferably above 2.8. Preferably, the apolar solvents are liquids that are water-miscible and are typically less polar than water.

Preferably the polar aprotic solvent is selected from the group consisting alkyl cyanides, ketones, organosulfur compounds, amides, ethers, more preferably from alkyl cyanides, most preferably acetonitrile.

Further preferred polar aprotic solvents that are useful for dissolving crudes containing epothilones include acetone, dimethyl sulfoxide, dimethylformamide and dioxane.

The polar aprotic solvents also include esters, wherein the esters are preferably acetyl esters, more preferably selected form the group consisting isopropyl acetate, n-propyl acetate, n-butyl acetate and t-butyl acetate or a mixture of any of at least two of the listed esters.

After dissolving a crude containing epothilones in a polar aprotic solvent, an effective amount of a protic anti-solvent is then added to the solution in order to induce crystallization of an epothilone. Upon addition of an effective amount of the anti-solvent, the resulting solution (including the solvent and the anti-solvent) exceeds the solubility limit of an epothilone thereby causing the epothilone to crystallize from the solution. Preferably epothilone B crystallizes from the solution, while other epothilones that may be present in the solution remain in the solution below their solubility limit.

Useful polar protic anti-solvents include solvents that are effective in modifying the solubility limit of epothilones, preferably of epothilone B, in the aprotic solvent solution. More particularly, an effective anti-solvent is miscible in the aprotic solvent solution by lowering the electric dipole moment of the solution.

The polar protic anti-solvents are polar solvents containing at least one hydrogen atom that is covalently bound to an electronegative atom, which leads to a pronounced intra-molecular polarization. Due to the intra-molecular polarization, molecules of protic solvents can participate in an inter-molecular hydrogen bonding, which is a strong intermolecular force. Preferred protic anti-solvents contain hydroxyl groups (—OH) and/or amino groups (—NH). Additionally, these O—H or N—H groups can serve as a source of protons (H+).

Preferably protic anti-solvents have moderate electric dipole moments below 2, more preferably below 1.9.

Preferably the protic anti-solvent is selected form water and lower alcohols (in particular C1 to C3 alcohols), preferably methanol, ethanol, and propanol (n-propanol and i-propanol). A particular preferred protic anti-solvent is water.

As aprotic solvents are not able to form inter-molecular hydrogen bonds, preferably protic and aprotic solvents used in the invention are miscible with each other in all proportion, but do not associate strongly with each other. Solvents are usually miscible when their solubility parameters, preferably their Hildebrand solubility parameters (a dimensionless parameter) do not differ by more than 5 units.

According to the invention, a total amount of a polar protic anti-solvent is added to the amount of said polar protic solvent in a ratio by volume of 12:1 to 1:3 (v/v), preferably in a ratio of 10:1 to 1:3 (v/v) to form slurry.

Preferably the volume of the protic anti-solvent that is added relative to the volume of aprotic solvent in the solution ranges from about 6:1 to about 1:3 (v:v), more preferably from about 5:1 to about 1:2 (v:v), most preferably from about 4:1 to about 2:1 (v:v).

In one embodiment of the invention, the crude containing epothilone is dissolved in an aprotic solvent, that already contains an amount of the protic anti-solvent up to a concentration of 0.1 to 10 percent by volume, more preferably 1 to 7 percent, especially 2 to 5 percent by volume.

Crystallization of the epothilone may be conducted using either a batch crystallization process or a continuous crystallization process.

In a batch process, the starting solution is prepared by dissolving a crude in an aprotic solvent in a single container where crystallization occurs, when adding the polar anti-solvent. After the crystals are isolated the process can be repeated.

In a continuous crystallization process, a solution is also prepared by dissolving a crude in an aprotic solvent in a single container. The resulting solution is pumped continuously into a crystallization vessel, wherein at the same time, the protic anti-solvent is pumped into the crystallization vessel in an amount that will result in the desired final anti-solvent concentration giving hereinabove. Crystallization occurs in this vessel, which is typically at a higher rate than in a batch vessel due to the continual presence of seed crystals. The product slurry is continuously pumped out of the vessel at a rate equal to the sum of the aprotic solvent and protic anti-solvent inflows, thus maintaining a constant volume in the crystallizer.

In both embodiments, the isolation of the crystalline solid is preferably carried out via filtration, preferably using a Büchner funnel, or by centrifugation, followed by a drying procedure to provide a final product.

Advantageously, solvents used in the process may be recovered by distillation. Dissolved epothilones in the supernatant can be recovered and/or either recycled.

Preferably the isolated crystalline solid is washed with water and dried, more preferably dried only. Advantageously, the processing is performed so that volatile solvent residues are removed to yield a high-quality epothilone.

Preferably the drying process is done by evaporation in a temperature range between 0 to 50° C., more preferably between 5 to 40° C., most preferably between 10 to 30° C. at a pressure of 1 to 1000 mbar, more preferably of 1 to 600 mbar, most preferably of 1 to 400 mbar.

Optionally, the solution is seeded with appropriate epothilone crystal nucleuses in a sufficient quantity, preferably less than 2.0 percent by weight, more preferably less than 1.0 percent by weight based on the total weight of the solution in order to promote the crystallization process and to enhance purity of the crystalline solid.

An appropriate epothilone crystal nucleus also called seed crystal is a small piece of single-crystal/polycrystal epothilone from which a large crystal of epothilone is to be grown, preferably with the same crystal modification.

Advantageously, seeding with appropriate epothilone crystal nucleuses can support epitaxial growth of single-crystal, which comprises epothilone with a defined crystal modification. A single-crystal is a highly ordered crystal, preferably characterized by only one crystal modification of an epothilone derivate.

Preferred alkyl cyanides used as polar aprotic solvent are miscible with water in all proportions, but do not associate strongly with water molecules and thus acetonitrile-water mixtures remain binary in character. Preferably alkyl cyanides having a total number of carbon atoms ranging from 2 to 10, more preferred from 2 to 8.

Preferably the alkyl cyanide is acetonitrile and/or propionitrile, most preferred acetonitrile.

The rate of addition of the protic anti-solvent to the solution containing a crude dissolved in an aprotic solvent can affect the purity of the epothilone crystals that are formed. For example, adding the protic anti-solvent in a single addition may cause the epothilone to crystallize rapidly from the aprotic solvent resulting in epothilone crystals that are lower in purity than would be formed if the protic anti-solvent is added to the solution slowly over time, or in multiple small portions.

Optionally the total amount of the protic anti-solvent is added in one addition. However, preferred is a stepwise addition over a maximum period of 24 h, more preferably of 20 h, most preferably of 16 h.

Preferably in a first step the protic anti-solvent is added to the solution up to a concentration of 0.1 to 10 percent by volume, more preferably 1 to 7 percent, especially 2 to 5 percent by volume.

Preferably, the protic anti-solvent is added to the solution containing a crude dissolved in an aprotic solvent while both solvents are maintained at about room temperature, preferably about 20±5° C.

Standard conditions are defined as 25° C. and 101.3 kPa. As used herein, the term “room temperature” refers to a temperature between 10° C. to 30° C., more preferably 15° C. to 25° C.

After addition of the protic anti-solvent, the resulting solution consisting of an aprotic solvent and a protic anti-solvent is allowed to stand, with mild to moderate agitation, at approximately room temperature so that the epothilone crystallizes from the solution. Typically, the epothilone crystallizes within about 24 h after the addition of the protic anti-solvent, more preferably within about 20 h, most preferably within about 16 h.

Preferably the crystalline solid is dried by evaporation.

Preferably in the crude the amount of epothilone B is in a molar ratio to the amount of epothilone A in the range of 10:1 to 1:2, more preferably of 6:1 to 1:1.

A crude containing epothilones can also contain impurities such as particulate materials, inorganic nutrients, sugars, organic acids, and amino acids. Preferably the amount of impurities in a crude is less than 5.0 percent by weight, more preferably less than 3.0 percent by weight based on the total weight of the crude.

However, a purification process according to the invention is suitable to separate impurities, so that in a crystalline solid the amount of impurities is less than 2.0 percent by weight, more preferably less than 1.0 percent by weight based on the total weight of the crystalline solid.

The method of the invention is used to produce a purified crystalline solid comprising epothilones, especially epothilone B and epothilone A in a molar ratio between 2:1 and 25:1, more preferably between 5:1 and 20:1. In a purified crystalline solid the molar ratio between epothilone B and epothilone A is at least twice as high as in the crude

The purified epothilones of the invention can be used as an active ingredient for the preparation of pharmaceutical composition.

The following examples serve to explain the present invention in more detail without representing a restriction of the scope of protection defined in the Claims.

Caution: When handling materials containing epothilones, appropriate protective measures must be taken, where necessary, in view of their high cytotoxicity.

FIG. 1: Chromatogram of epothilone crude as starting material.

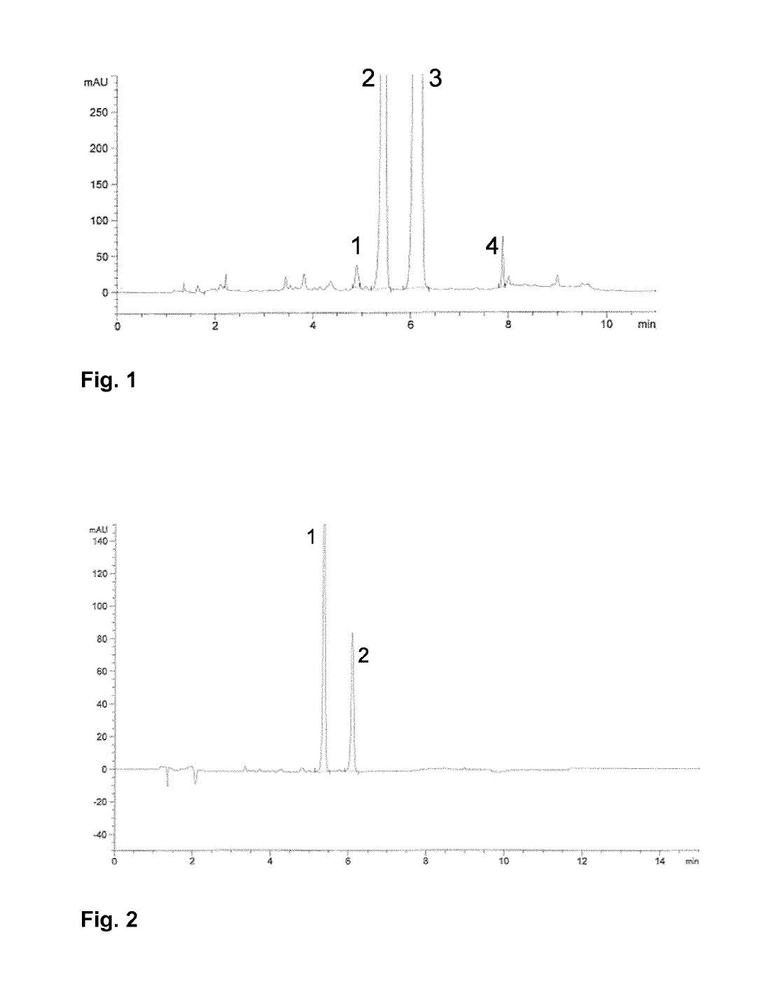

FIG. 2: Chromatogram of a mother liquor, wherein the ratio of epothilone B and A is 1:1.84.

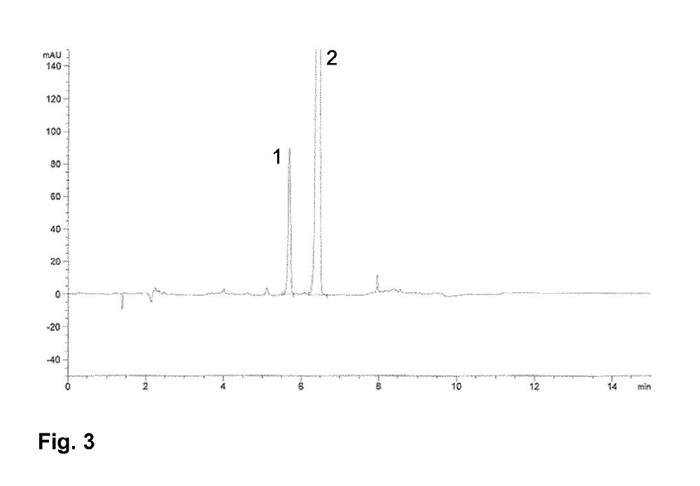

FIG. 3: Chromatogram of isolated epothilone, wherein the ratio of epothilone B and A is 12.7:1.

### EXAMPLE 1

### Crystallization in Acetonitrile/H₂O

In a first step 0.5 g of a crude containing epothilone A and epothilone B in a molar ratio of 1 to 3 is dissolved in 5 mL acetonitrile at room temperature giving a slightly turbid solution. After addition of two drops of water the solution is clear. Over a period of 40 min, 50 mL water are added to the solution while precipitation occurs. The mixture is stirred at room temperature (22° C.) for 2 h and the solid is isolated via filtration (G3 Büchner funnel). After drying, 0.69 g of a white crystalline solid is isolated, wherein epothilone B and epothilone A are in a molar ratio of 10 to 1. The mother liquor contains epothilone B and epothilone A in a molar ratio of 1 to 2.

### EXAMPLE 2

### Crystallization in Acetonitrile/H₂O

1.3 g of a crude containing epothilone A and epothilone B in a molar ratio of 1 to 3 is dissolved in 15 mL acetonitrile at room temperature (23° C.) giving a slightly turbid solution. After addition of 0.5 mL water the solution is clear and 15 mL of water are added to the solution in three equal portions.

After the first portion of water no precipitation is observed. The second portion leads to a slightly turbidity of the solution, while after addition of the third portion a thick suspension is formed. The mixture is stirred at room temperature for 1 h. As the mother liquor still contains a lot of epothilone B (ratio B/A=2 to 1), an additional 5 mL portion of water is added to the suspension.

The mixture is stirred at room temperature (23° C.) for 16 h and the solid is isolated via filtration (G3 Büchner funnel). After drying, 0.50 g of a white crystalline solid is isolated, containing epothilone B and epothilone A in a molar ratio of 17 to 1.

### EXAMPLE 3

### Crystallization in Acetonitrile/H₂O

3.0 g of a crude containing epothilone A and epothilone B in a molar ratio of 1 to 3 is dissolved in 30 mL of an acetonitrile/water solution (95 to 5) at room temperature (21° C.) giving a clear solution. Precipitation occurs, when adding 41 mL of water slowly and continuously. The solution contains epothilone A and epothilone B in a molar ratio of 1 to 1.16.

Additional 15 mL of water are added slowly, after which the solution contains epothilone A and epothilone B in a molar ratio of 1 to 1.59. Afterwards an additional portion of 15 mL water is added slowly, wherein the solution contains epothilone A and epothilone B in a molar ratio of 1 to 1.84.

After adding 15 mL water, the mixture was stirred at room temperature (22° C.) for 16 h and the crystalline solid is isolated via filtration (G3 Büchner funnel). The crystalline solid is dried by evaporation for 60 h, 200 mbar at room temperature and finally 3 h, 1 mbar at room temperature. After drying, 2.3 g of a white crystalline solid is isolated, containing epothilone B and epothilone A in a molar ratio of 12.7 to 1. The mother liquor contains epothilone B and epothilone A in a molar ratio of 1 to 2.17.

### EXAMPLE 4

### Crystallization in Acetonitrile/H₂O

12.4 g of a crude containing epothilone A and epothilone B in a molar ratio of 3 to 1 is dissolved in 180 mL acetonitrile/water solution (95 to 5) at room temperature (21° C.) giving a clear solution. Afterwards 300 mL water was added in 30 min. After the addition of 80 mL precipitation occurs. After complete addition the mixture was stirred at room temperature (22° C.) for 16 h and the crystalline solid is isolated via filtration (G3 Büchner funnel). The crystalline solid is dried by evaporation for 60 h, 200 mbar at room temperature and finally 3 h, 1 mbar at room temperature. After drying, 9.0 g (yield: 88%) of a white crystalline solid is isolated, containing epothilone B and epothilone A in a molar ratio of 11.4 to 1.

### EXAMPLE 5

### Analytical Method

For the determination of the ratio between epothilone A and epothilone B HPLC analysis is used. The method is developed as IPC analysis for the fermentation process (SOP 61.321 version 7.0).

The column used for the HPLC experiment was the Pack Pro C18 column, 4.6 mm×150 mm, 3 μm particle size. Separations is achieved at 50° C. using Solvent A (Na₂HPO₄ buffer pH3.5/acetonitrile=90 vol %/10 vol %) and Solvent B (acetonitrile) with a simple isocratic flow at a rate of 1.5 mL/min. The wavelength for detection is 250 nm, and the injection volume of each sample is 10 μL.

Sample Preparation Solid:

5 mg of the solid is dissolved in 20 mL of a 1/1 mixture of acetonitrile and water.

Sample Preparation Mother Liquor:

300 μL of the mother liquor is dissolved in 20 mL of 1/1 mixture of acetonitrile and water.

FIG. 1 exhibits a chromatogram of an epothilone crude, wherein the ratio between epothilone A and epothilone B is 1 to 3.2.

| Nr. | Retention time [min] | Area [%] | Name |
|---|---|---|---|
| 1 | 4.90 | 0.39 | Impurity |
| 2 | 5.46 | 23.48 | Epothilone A |
| 3 | 6.16 | 75.62 | Epothilone B |
| 4 | 7.89 | 0.50 | Impurity |

After the purification process from the graph in FIG. 2 the ratio between epothilone A and epothilone B in the mother liquor is calculated to be 1.84 to 1.

| Nr. | Retention time [min] | Area [%] | Name |
|---|---|---|---|
| 1 | 5.38 | 64.81 | Epothilone A |
| 2 | 6.11 | 35.19 | Epothilone B |

From the chromatogram in FIG. 3, for the isolated crystalline solid a ratio of epothilone A and B is calculated to be 1 to 12.7.

| Nr. | Retention time [min] | Area [%] | Name |
|---|---|---|---|
| 1 | 5.70 | 7.28 | Epothilone A |
| 2 | 6.43 | 92.72 | Epothilone B |

1. A method of separating an epothilone from a mixture containing epothilone B, comprising the steps:
a. dissolving a crude containing epothilones in an polar aprotic solvent,
b. adding an amount of a polar protic anti-solvent in a ratio to the amount of said polar aprotic solvent of from about 12:1 to 1:3 (volume/volume) to form a slurry, and
c. isolating a crystalline solid containing epothilone B from the said slurry.

2. A method according to claim 1, wherein the polar protic anti-solvent is water.

3. A method according to claim 1, wherein the epothilones are selected from epothilone A and epothilone B.

4. A method according to claim 1, wherein the aprotic solvent is an alkyl cyanide.

5. A method according to claim 2, wherein in the crude comprises epothilone A and epothilone B, and the amount of epothilone B is in a ratio to the amount of epothilone A in the range of 10:1 to 1:2.

6. A method according to claim 1, wherein the solution is seeded with ephothilone crystals.

7. A method according to claim 4, wherein the alkyl cyanide is acetonitrile.

8. A method according to claim 1, wherein the amount of the polar protic anti-solvent is added stepwise over a maximum period of 24 h.

9. A method according to claim 1, wherein the crystalline solid is dried by evaporation.

10. A method according to claim 1, wherein the crystalline solid contains epothilone B and epothilone A in a molar ratio between 2:1 to 25:1.

11. The method of claim 1, wherein the isolated crystalline solid comprises epothilone B and epothilone A in a molar ratio of 2:1 to 25:1.

12. A pharmaceutical preparation comprising an isolated crystalline solid containing epothilone B obtained by the process of claim 1.
